(19) United States
(12) Patent Application Publication
(10) Pub. No.: US 2014/0364701 A1
(43) Pub. Date: Dec. 11, 2014

(54) APPARATUS AND METHOD FOR ASSESSING FUNCTIONAL STATE OF BODY SYSTEMS INCLUDING ELECTROMYOGRAPHY

(71) Applicant: Leonid Masakov, Espoo (FI)

(72) Inventor: Leonid Masakov, Espoo (FI)

(21) Appl. No.: 13/912,176

(22) Filed: Jun. 6, 2013

(51) Int. Cl. A61B 5/0488; A61B 5/0432; A61B 5/0476

(52) U.S. Cl. CPC A61B 5/0488; A61B 5/0476; A61B 5/0432
USPC 600/301; 600/483

(57) ABSTRACT

An apparatus and method for assessing the functional state of a user. Interface logic preferably executing on a mobile device permits the accumulation of bio-signals from a user. The bio-signals may be assessed to determine the functional state of various body systems. The assessments may be performed by processing logic located at a distance from the user to permit multiple assessments to happen at the same time. Electromyography is used for muscle assessment to permit assessment without muscle exhaustion. Bio-signal propagation is preferably wireless. Various embodiments are disclosed.

### CROSS-REFERENCE TO RELATED APPLICATION/PATENT

This application is related to U.S. patent application Ser. No. ______, entitled Apparatus and Method for Functional State and/or Performance Assessment and Training Program Adjustment by Nasedkin, V., and filed on Jun. 6, 2013 which is hereby incorporated by reference as though disclosed herein. This application is also related to U.S. Pat. No. 6,572,558 issued to Masakov, et al. for an Apparatus and Method for Non-Invasive Measurement of Current Functional State and Adaptive Response in Humans which is hereby incorporated by reference as though disclosed herein.

### FIELD OF THE INVENTION

The present invention relates to efficiently and conveniently assessing the functional state of a subject under test (SUT) and, more specifically, doing this assessment in a manner that does not deplete skeletal muscles of the SUT or adversely affect recovery from injury, etc. A primary use of the present invention is to improve physical fitness training.

### BACKGROUND OF THE INVENTION

FIG. 1 is a reprint of FIG. 1 of U.S. Pat. No. 6,572,558 issued to Masakov, et al. for an Apparatus and Method for Non-Invasive Measurement of Current Functional State and Adaptive Response in Humans ('558 patent). While the system of FIG. 1 represents an advancement in the art of “functional” or “physical” state assessment, it is disadvantageous in many ways. The disadvantageous aspects include, but are not limited to, the following.

As shown in the figure, the system of the '558 patent uses a large number of wires to connect a subject under test (SUT) to the assessment equipment. This jumble of wires is cumbersome and problematic. A need thus exists to reduce or eliminate these wires. The present invention may include reducing the number of electrodes required for a given test or communicating wirelessly or both, among other techniques, to reduce clutter and aid in the efficient collection of data.

The '558 system utilizes a “JUMP TEST” to assess the state of skeletal muscles. To perform the jump test, a sensor mat 39 is provided and a SUT jumps as high and as often as they can for 10 or 60 seconds. While beneficial in collecting certain types of data, one disadvantageous aspect of the Jump Test is that it is fairly vigorous and may deplete the muscle(s) under test, this is particularly true with the 60 second jump test. This vigorous, depleting test cannot be done everyday (without affecting athletic performance) and it cannot be done if a person is recovering from an injury or other disability. However, frequent assessing muscle state, for example, before or after daily workouts is very important to determine the appropriateness or effectiveness of a given training regime or preparedness for competition.

Thus, a need exists for assessing the functional state of a muscle without significantly depleting the muscle. A need also exists for assessing the muscles of a person who is recovering from injury or who has a disability. Furthermore, if the test is less depleting, for example, as is the test of the present invention, then a SUT is more likely to undergo the assessment.

FIG. 1 shows a one-to-one ratio between the data collection equipment (on the left side of the figure) and the data processing equipment (on the right side of the figure). In this arrangement, a SUT has to come to the location of the data collection equipment, rather than undergo assessment where they are. Furthermore, the processing logic is more expensive than the sensor equipment, so it would be cost effective to have many data collection units per processing units.

The present invention permits a SUT to do an assessment (data collection) wherever they are, and to reduce the cost of the assessment by having fewer data processing units relative to data collection units.

While the technology of the present invention may be used by individuals training alone, it has particular benefit to teams. In the context of a team, if the system of FIG. 1 is used, each team member would go to the location of the test equipment and the athletes would be tested one after the other. This could take quite a bit of time. For example, at 10 minutes a test for a 60+ member football team, the assessment would take at least 10 hours, using one system. If this is done each day to assess the benefit of a given training regime, the amount of time needed for assessment quickly becomes impractical, or the assessment becomes undesirably expensive if many systems are purchased and maintained for parallel assessments.

A need exists to permit team members to individually collect data where they are, when they can (for example, when they wake up in the morning), in a reasonable amount of time and without over-exerting themselves, and to transmit that data to a centralize processor for efficient assessment by trained professionals. The sensed data can be processed and individual and/or team training regimes modified, potentially daily, to provide optimum training and optimum readiness for performance.

While beneficial for team assessment, the rapidness and ease of assessment, etc., of the present invention is also beneficial to the non-team athlete and/or the individual merely trying to have improved health and performance.

### SUMMARY OF THE INVENTION

Accordingly, it is an object of the present invention to provide assessment of a SUT's current functional state and/or adaptive response state in a manner that does not require significant physical exertion by the SUT.

It is another object of the present invention to utilize electromyography (EMG) to assess the condition or state of a SUT's neuro-muscular system.

It is another object of the present invention to combine electromyography assessment with other cardiac and/or brain wave assessment to achieve a clearer picture of the functional state and/or state of adaptive response of a SUT.

It is yet another object of the present invention to provide this assessments in a non-invasive manner, with a reduced electrode count, and/or in a wireless or substantially wireless environment.

These and related objects of the present invention are achieved by use of an apparatus and method for assessing functional state including electromyography as described herein.

The present invention includes both apparatus and method embodiments of carrying out these and related features.

The attainment of the foregoing and related advantages and features of the invention should be more readily apparent to those skilled in the art, after review of the following more detailed description of the invention taken together with the drawings.

### BRIEF DESCRIPTION OF THE DRAWINGS

FIG. 1 is a diagram of the prior art system of the '588 patent.

FIG. 2 is block diagram of a system for assessing functional state including electromyography in accordance with the present invention.

FIG. 3 illustrates an embodiment of a sensor support belt and transmitter in accordance with the present invention, while FIG. 4 illustrates a sensor in the belt of FIG. 3.

FIGS. 5 and 6 illustrate a transmitter in accordance with the present invention.

FIG. 7 is a flow diagram for performing cardio, brain and EMG assessment on a SUT using the system of FIG. 2.

FIG. 8 illustrates one embodiment of results and textual conclusions from a 2-lead DECG assessment.

FIG. 9 is a flow chart for an electromyography assessment in accordance with the present invention.

FIGS. 10A-D illustrate electrode placement for EMG assessment.

FIG. 11 illustrates a sensed EMG signal.

FIG. 12 illustrates one embodiment of a simplified presentation of test results for individual body system test.

FIG. 13 illustrates one embodiment of a simplified presentation of combined assessment results.

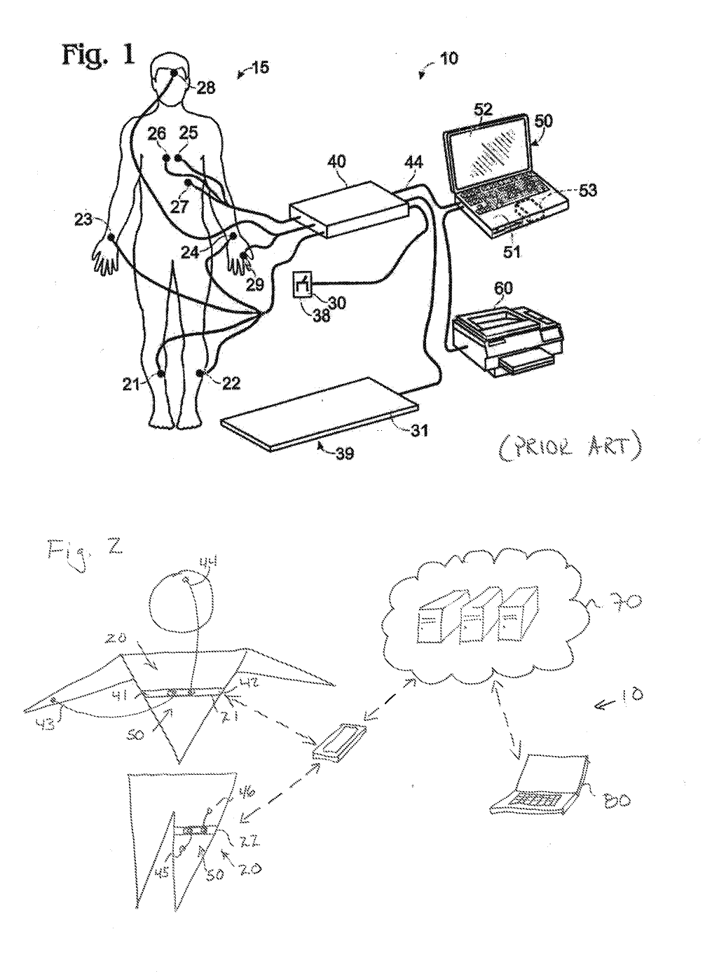

### DETAILED DESCRIPTION

Referring to FIG. 2, a diagram of a functional state assessment system 10 in accordance with the present invention is shown. System 10 may include one or more belts 21,22 onto which sensors 41-46 and a transceiver or transmitter pod 50 are coupled, a hand held interface device 60, and a remote processor 70. The remote processor may be connected via a mobile device network (e.g., cellular phone network), the internet and/or both, and may be referred to as a “cloud” computer or processor, though it may be otherwise networked without deviating from the present invention. A third party communications device 80 may also be included to enable a trainer, health professional or other party to review the results, configure tests, etc., or for a system administrator to perform maintenance and support.

Belts 21,22 may vary depending on the part of the body to which they are attached, to accommodate different tests or electrode configurations and/or for aesthetic or structural purposes, etc. They may be referred to generally with reference numeral 20 or specifically with their reference numerals 21,22. While the term “belt” is used, it should be recognized that the function is to hold the transceiver and/or sensors in an appropriate or convenient place, and other structures such as a strap, harness, direct attach electrodes, or other, may be used without deviation from the present invention. Note that if direct-attach wireless electrodes are used, these electrodes may communicate directly to the mobile device without transceiver pod 50.

FIG. 3 illustrates one embodiment of a belt 20 (belt 21 of FIG. 2). The belt includes a band 23 having first and second ECG sensors 41,42 affixed to (or formed integrally therewith). FIG. 4 is a close-up of a sensor 41,42. These sensors are preferably positioned on the inward side of belt 20 so that they make contact with the chest of a SUT, preferably on the far left and right sides.

A conduit 24 couples sensors 41,42 to fastening snaps or clips 25,26 respectively. The fastening snaps are preferably made of a conductive material and preferably serve (a) to releasably dock the transceiver pod 50 and (b) as an electrical conduit for conducting sensed bio-potentials from sensor 41,42 to pod 50.

Band 23 is preferably formed at least in part of an elastic material so that the electrodes 41,42 are held in contact with a user's skin to assure good cardio signal capture. Band 23 may have a size adjustment member 28 and/or an open-and-close buckle 29, though it may be sufficiently elastic or made in different sizes so that one or more of these items are not needed. Note also that belt 22 in the EMG configuration need not have sensors 41-42.

FIG. 4 illustrates one embodiment of sensor electrodes 41,42 on the inside of belt 20,21. The sensor electrodes may include a conductive mesh that is affixed to band 23 and coupled to conduit 24. Other sensor types may be used without departing from the present invention. Sensors 41,42 are involved in cardiac bio-potential or bio-signal measurement, the type of biological signals that can be used for heart rate, heart rate variability, ECG and DECG measurements, etc.

FIGS. 5 and 6 are a front view and a top view of pod 50. Complementary snaps 55,56 (visible in FIG. 6) extend off the back of transceiver pod 50 for releasable coupling to clips 25,26 on the belt. Supplemental snaps 57,58 may also extend from the pod, and may be used for brain wave sensors 43,44 or EMG sensors 45,46, though they may be dedicated to a specific sensor type (as discussed below).

The supplemental snaps may be universal (accepting leads of different types of electrodes) or dedicated, i.e., the snap formed complementary with a lead to an electrode designed for a specific bio-signal type. To support brain wave and EMG assessment, there may be four supplemental snaps, two supplemental snaps complementary with leads 43-44 for a brain wave test and two supplemental snaps complementary with leads 45-56 for an EMG test. This would make the pod “idiot-proof” with respect to attaching different types of sensors (i.e., assuring that a sensor is connected to its corresponding transmission channel).

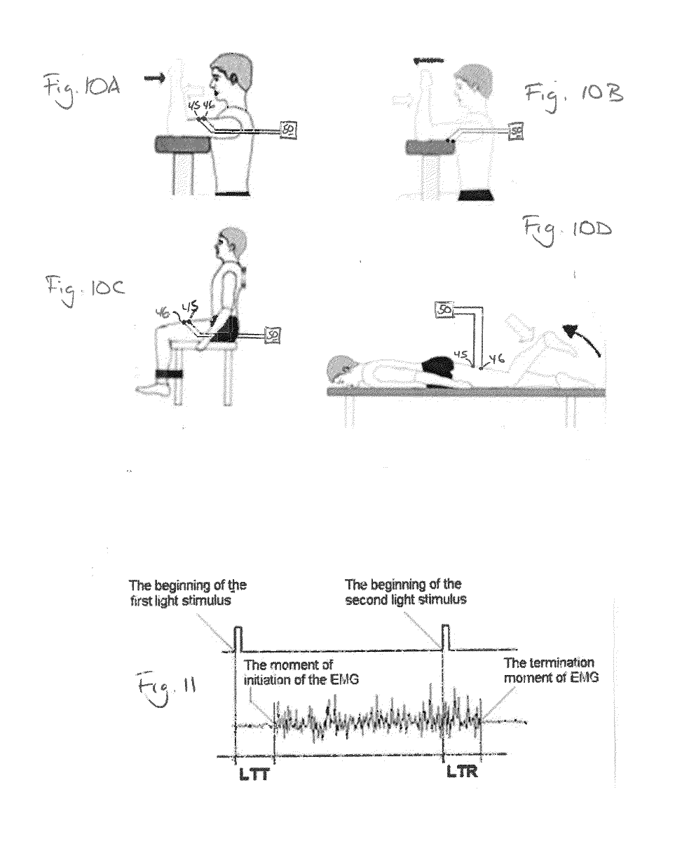

To assess the state of skeleton muscles, a belt 22, like chest belt 21 or different, for example, smaller and without chest electrodes 41-42, etc., may be used. This belt may be placed around or in proximity to the skeletal muscle to be assessed. Electrodes 45,46 may be snapped onto pod 50 and used for an electromyography (EMG) measurement, as discussed below. While attachment to the front of the thigh (ie, quadriceps muscle) is shown in FIG. 2, the EMG sensor arrangement could be connected to other skeletal muscles as discussed below with reference to FIGS. 10A-10D.

Hand held device 60 may be a mobile phone, tablet computer or other lightweight, low-power electronic communication device. In one embodiment of the present invention, device 60 includes a graphic user interface programmed to allow the user to select various functional state tests and, in turn, to instruct the SUT on how to conduct selected tests—where to place electrodes, whether/when to rest or contract a given muscle, or something else.

Sensed data for a given test is transmitted, preferably wirelessly, from pod 50 to hand-held device 60 using Bluetooth or other known technology. This removes the jumble of wires used in the '558 patent. From there, the sensed bio-signals may be transmitted to processing computer 70. Sensed data is assessed on the cloud server and test “results” transmitted back to the SUT and displayed on mobile device 60.

Third party device 80 may be configured along with the cloud server 70 to allow a third-party to access results/data, modify test parameters, and/or conduct maintenance and support, etc.

Transmission logic 51 is pod 20 preferably has transmission channels that support transmission of bio-signals for the cardiac, brain and skeletal muscle sensors to mobile device 60. In one embodiment, there may be multiple pods 50, each with a transmission channel unique to a type of bio-signal, i.e., one each for cardiac, brain and EMG. In another embodiment, the pod may have multiple transmission channels within it, each for a given type of bio-signal, and selection logic to propagate a specific type of bio-signal at a given time (for transmission through the appropriate channel to a transmit antenna in the pod).

In a selection based multi-channel embodiment, the transmission logic preferably responds to a signal from mobile device 60 that indicates the type of bio-signal to transmit. The transmission channels may include filters, amplifiers and/or converters as known in the art. Multiplexing or other suitable channel selection may be used for channel selection.

### Table I

### Body System Tests

Tests Body System Examined

### 1. Electromyogram (EMG) Neuro-Muscular

### 2. Heart Rate Variability Cardio System

### 3. ECG, Differential ECG Metabolic

### 4. Omega Wave Circulation, Detox, Adrenal, CN

Table I lists tests and the corresponding body systems that are assessed by the specific test. System 10 permits ready assessment of these body systems which together give a comprehensive view of the functional state of a SUT. A representative assessment in now described.

Referring to FIG. 7, a flow diagram of interfacing and processing for data collection form a SUT is shown. Mobile device (MD) 60 preferably has interface logic (IL) 61 that enables a SUT to perform an assessment. IL 61 may take the form of an “app” that is downloaded and executed on mobile device 60. IL 61 prompts a SUT for the type of assessment or assessments the SUT desires. IL 61 may be tied to the MD's calendar system and even prompt the SUT at a given time or day to begin assessment. The SUT may select the desired test. For teaching purposes, it is assumed the SUT selects cardiac based assessment first, followed by brain and EMG, though individual tests, or other combinations, or a different order may be selected.

In response to cardio-based assessment selection by a SUT, IL 61, via the screen 65 on MD 60, instructs the SUT where to place the sensors and awaits a “Sensors Placed Acknowledgment” from the SUT (111).

A handshake test (113) may then be conducted to assure that the appropriate signals type and magnitude is being received from the sensors 41-42 and pod 50. If not received, the SUT is so informed and prompted to correct. Next, the SUT is given any necessary instructions for the test, for example, to relax, or lie down or other instructions and also given an option to start the test (115). A visible count down to test start may be displayed (117) and then sensor data collection is commenced for a predefined time period (119). The collected data is preferably tagged with time, test type, user, and/or other information (121). IL 61 may determine if the type, quality and quantity of data is sufficient (step 123) and if the data collection has been successfully performed. If so, the SUT is so informed (step 125). If not, the SUT is informed and asked to repeat the collection process. Depending on the type of results (magnitude and form of data received), specific instructions on how to correct issues that may have lead to incomplete data collection is fed back to the SUT (127). The collected tagged data may be transmitted to cloud processor 70 or held for transmission with the collected data from the other tests (step 129).

The middle column of FIG. 7 represent brain wave data collection and, more specifically, omega wave sensing. A similar procedure is performed here. The SUT is instructed on how to place the sensors (141) and a handshake is performed (143) to assure connection to brain wave sensors 43-44 (through pod 50 or otherwise). The SUT is then instructed on how to perform the test (115). In the omega wave test, a SUT preferably starts at rest (sitting, standing, lying down as instructed), then is instructed to perform an act, e.g., to generate a “load,” such as clenching a first or a deep knee bend (147). The Resting Potential and After Load potential are recorded (149). This data is appropriately tagged (151) and a determination is made as to whether the test completed successfully (153). If so, a test successful message is displayed. If not, a trouble shoot and retry message is generated (157). The SUT may next be prompted for transmission to hold or transmit the collected data (159).

Referring to the right column of FIG. 7, a flow diagram of EMG data collection is presented. A SUT is prompted with instructions and requests similar to those followed in the cardiac and brain bio-signal assessments described above. For example, the EMG protocol may involve: sensor placement acknowledgment (171), handshaking for signal affirmation (173), instructions for test protocol and test instigation (175), test count-down and duration indication (177), data collection (179), data tagging (181), test successful determination (183), success notification (185), failure notification and correction/retest (187) and hold/transmit (189).

At step 189, transmit, the SUT may transmit the collected data to the processing computer 70. If at step 129 and 159, the collected data had been held, then all of the collected data may be bundled at step 189 and sent to processing computer 70. Computer 70 preferably conducts functional state assessment of the body systems in Table I based on the collected data. These assessments may be conducted as follows.

### Heart Rate Variability (HRV) Test—Cardiac

The heart rate variability test (HRV) is designed to give an indication of the state of the biological systems that regulate cardiac activity. The cardiac system functions best when it is regulated by the autonomic circuit. When homeostasis is broken (unbalanced) higher levels of the central regulatory system dominate cardiac activity. These changes in regulation are reflected in the variability of the heart rhythm. Processing cardiac signals as discussed below permits quantitative and qualitative analysis of the functional state of cardiac activity.

The following is a representative HRV test. It should be recognized that HRV tests that differ from that taught below are within the present invention when similar or producing similar results or when provided with one or more of the other types of tests taught herein.

In general, an HRV test conducted via system 10 records sensor data, constructs charts or “grams” (i.e., scatter-grams, histograms, frequency spectrum-grams, etc.) that reflect the sensed data, calculates indices from the grams and data, and performs rules based analysis of the indices values to generate signals representative of textual or graphic conclusions of the functional state of cardiac activity. These signals, once received by the MD 60 or the 3P computer 80, may be viewed by the SUT or 3P, respectively. FIG. 6 of the '558 patent illustrates representative HRV test results which may include a cardiogram, the above-mentioned charts/grams and textual conclusions of functional state.

The HRV test is based on the registration of cardiac contractions of standard electrocardiogram (ECG) readings over the course of a fixed span of time. For an assessment, belt 21 may be worn by a SUT such that the sensors touch the left and right side of the chest. The electrode arrangement is suitable for measuring RR intervals, thought alternative sensor placement may be utilized without deviating from the present invention.

The test records the change of period length (in seconds) between each cardiac contraction which is the time between ECG spikes, which are often designated with the letter R in an electrocardiogram.

Cardiac muscle electrical activity is recorded for a fixed time period, e.g., 120. A fixed number of consecutive heart beat intervals (RR intervals), e.g., 100, is selected and analyzed. The intervals are processed in this preferred method using a fast fourrier transformation to achieve frequency spectrum analysis and the density of interval frequencies is plotted in a frequency spectrum-gram 191. Frequency spectrum analysis is known in the art. Relevant frequency ranges include: high frequency=0.15 to 0.4 Hz; low frequency=0.04 to 0.15 Hz; and very low frequency=0.004 to 0.04 Hz, and signals representative of the collected data may be propagated to MD 60 or 3P computer 80 for display.

Various preferred indices for cardio system performance are respectively calculated based on frequency spectrum and other data and these include:

Vagus (parasympathetic) Regulation (VR);

Humoral Regulation (HR);

Sympathetic Regulation (SR);

Stress Index;

Share of aperiodic influences;

Standard deviation; and

Frequency of Cardiac Contractions (FCC).

Calculation of these or related indices is known in the art. (See Baevskiy, R. M., et al., Mathematical Analysis of Changes in Heart Rate Rhythm Under Stress, Moscow Science, 1984).

These indices are interpreted to generate textual or graphic conclusions about the functional state of cardiac activity. Condition statements are preferably generated for at least:

1. type of rhythm;

2. type of regulation of rhythm; and

3. type of vegetative homeostasis.

The type of rhythm is the heart beat rate. Type of regulation is based on VR (related to a norm) and conclusions may include sinus arrythmia (which is normal), stable rhythm, pace-maker dysfunction, etc. Type of vegetative homeostasis is based on HR, VR, and SR and reflects an evaluation of the balance between parasympathetic and sympathetic regulation of the heart. The indices may also be used to generate other conclusions about the functional state of the cardiac system including degree of stress of the regulatory mechanism (from normal to state of dysfunction), reserve status (from high to very low), readiness of system for loads (from optional to severe cardiac dysfunction demanding immediate cardiology consultation) and adaptation to external influences (from stable to breakdown in adaptation).

### Differential ECG (DECG) Test—Metabolism

The heart is a cardiac muscle and energy metabolism in the heart can be monitored with an ECG. Since there is a known correlation between energy metabolism in cardiac muscles and in skeletal muscles, conclusions about the state of skeletal muscles can be drawn from analysis of cardiac muscle energy metabolism.

For this assessment, belt 21 is positioned such that electrode 42 is placed in the V6 position of a 6-lead configuration. This two-electrode arrangement allows the replacement of a multi-electrode/lead configuration with the simple two-electrode belt solution. Furthermore, it affords a higher correlation with metabolic parameters, particularly aerobic state. With respect to DECG, the sensed ECG signals are differentiated as discussed below to render a signal that is referred to herein as a DECG signal. It should be noted that this may not be a classical-type DECG signal based on Wilson positioning. However, the non-classical arrangement of FIG. 2 provides a desired trade-off of DECG-like data (and the benefits that come therefrom) with a simplified two-lead approach. The DECG of this embodiment of the present invention may be referred to as differentiated ECG from two electrodes placed on the rib cage. Notwithstanding the specificity of this preferred embodiment, it should be recognized that other electrode arrangements may be used without departing from the present invention, particularly those with a modified classical approach that result in use of fewer electrodes/leads and/or from which an ECG measurement is derived that is subsequently differentiated for processing/assessment.

For the ECG/DECG assessment, ECG data is recorded from each sensor 41-42 for a predefined time period, e.g., 120 seconds. The received ECG signals from the chest sensor electrodes are preferably differentiated and analyzed. A subset, e.g., 10-60 (30 in the present example), of consecutive QRS complexes (peak and recovery of differentiated heart beat contraction) are analyzed and R and S values are ascertained.

Indices for the representative DECG test are generated from the sensed data (preferably including averaged R and S values). These indices include the anaerobic power index (API) which is the magnitude of maximum oxygen consumption, VO2 max, the alactic capacity index (ALCI), the lactic capacity index (LCI), the anaerobic capacity index (ACI), the aerobic efficiency index (AEI), and the system adaptation index (SAI). Calculation of these or related indices is known in the art. (See publications of Kiev Sports Medicine University by Beregovog, V. Y., or Dushanin, S. A. (1986)).

These indices are then analyzed (step 220) to generate textual conclusions about the functional state of the metabolic system. This analysis is preferably carried out using a rules-based analysis as discussed below. The generated condition statements preferably address:
1. state of functional reserves;
2. speed of recovery process;
3. resistance to hypoxia (oxygen debt); and
4. aerobic reserves.
   Each of these items may range from high to low and the generate textual conclusions preferably state the corresponding level.

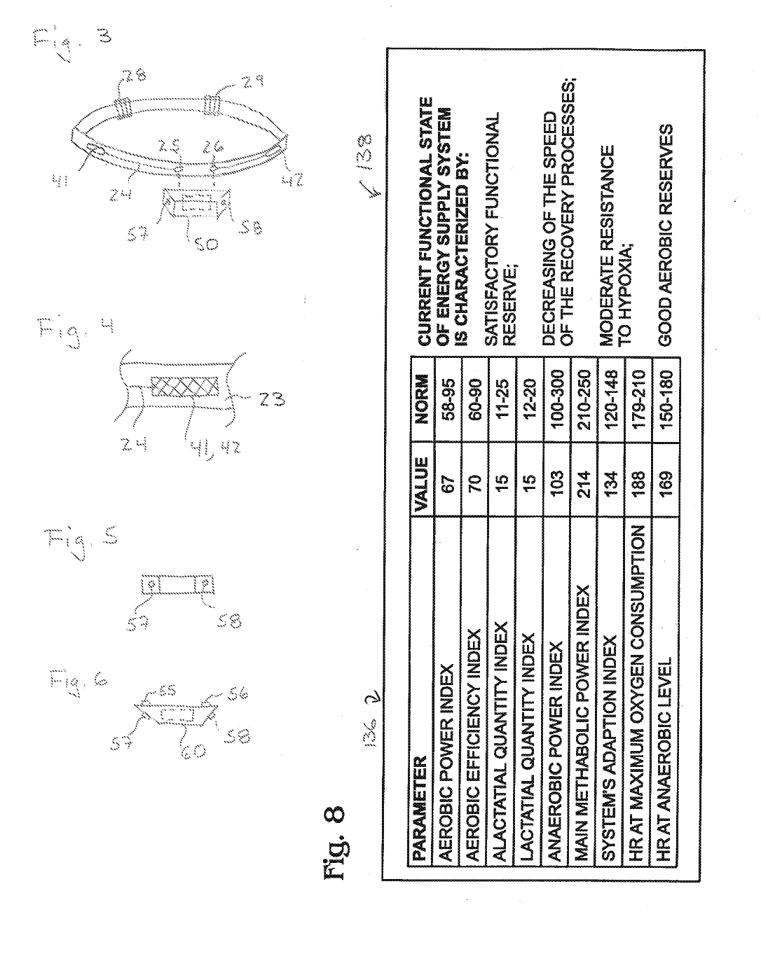

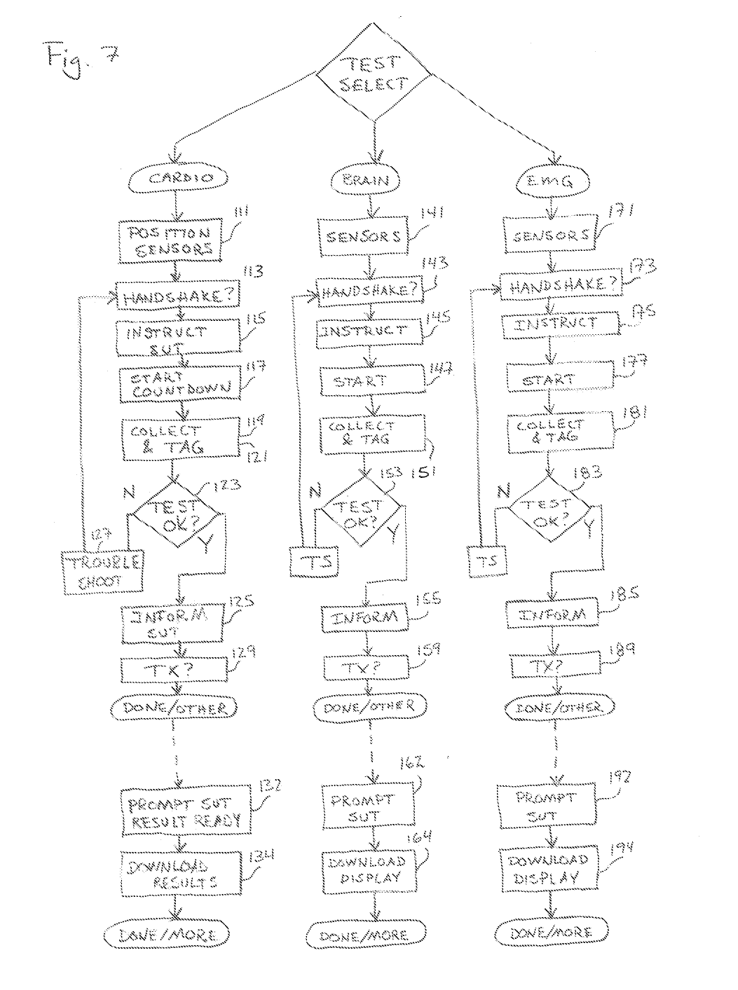

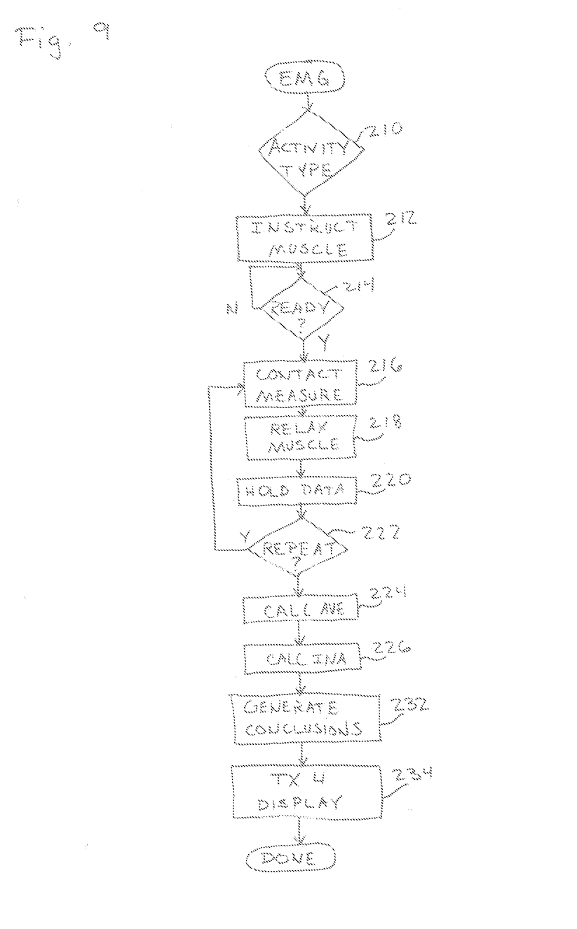

The indices and textual conclusion are depicted in FIG. 8 with reference numerals 230 and 235, respectively.

### Omega Wave (OW) Test—Circulatory, Detox, Hormonal, CN

Omega brain waves and omega brain wave potential, particularly the DC potential, have been shown to have a relationship to the performance of the central nervous, circulatory, detoxification and hormonal systems.

The following is a representative omega wave (OW) DC potential test. It should be recognized that tests that differ from that taught below are within the present invention when similar or producing similar results or when combined with one or more of the other tests taught herein. The OW test results may be presented using charts of resting omega potential v. time, post-load omega potential v. time and textual conclusions of functional state, among other parameters/results. Representative charts and graphic and/or textual conclusions are shown in FIGS. 10-11 of the '588 patent, cited above.

The base omega potential at rest has been identified as an indicator of the level of the functional state of the central nervous system and its adaptive reserves. Three levels of base omega potential have been empirically differentiated in healthy people and these are low level (<0 mV), medium level (0-40 mV), and high level (41-60 mV). Low level is characterized by a lowered level of wakefulness, quick exhaustion of psychic and physical functions, unstable adaptive reactions and limited adaptive potential. Medium level is characterized by an optimal level of wakefulness, high stability of psychic and physical functions, sufficient adaptive potential and stable adaptive reactions. High level is characterized by a state of psychic-emotional tension, high stability in response to loads and adequate adaptive reactions.

Iberal and McCullock have shown in their research that there is a time scale for turning on various system resources in response to a stress (i.e., post-load potential). Empirical data has shown that the dynamics of omega potential after an external stress are closely related to the dynamics of various body system processes being turned on. As a result, three time zones of omega potential change, after a single stress load, have been identified and they are Zone A (0-1.5 minutes), Zone B (1.5-4 minutes), and Zone C (4-7 minutes). Zone A characterizes the functional state of the cardio-respiratory (circulatory) system. Zone B characterizes the functional state of the detoxification system (i.e. gastro-intestinal tract, liver and kidneys, etc.). Zone C characterizes the functional state of the hypothalmic, hypophysial and adrenal glands (hormonal system).

The omega wave test is preferably conducted with chlorine-silver weak-isolating 43-44 electrodes. The electrodes are placed on the test subject (one at the center of the test subject's forehead and one at the base of the right thumb) while the test subject is either sitting or lying in a state of rest.

In step 149 (FIG. 7), MD 60 indicates to the SUT that the test in commencing and begins collecting sensed omega wave potential from the SUT. These signals are preferably recorded for a pre-defined time period, preferably approximately seven minutes, after which a test end signal is generated. The base potential provides a base line from which to assess post-load potential.

To perform the post-load assessment, a SUT is prompted via MD 60 to undertake a physical load such as one or two rapid knee bends. The omega potential of the SUT is recorded for a fixed period of time, approximately seven minutes, after which an end test signal is generated. A graphic representation of the results of the post-load test is preferably generated and plotted (FIG. 10, '558 patent). The base and post-load potentials are then compared in each zone and textual conclusions are generated based on the percent difference between the base and post-load potentials.

In Zone A (circulation), the textual results preferably indicate a state ranging from significant hyperfunction to normal to significant hypofuntion.

In Zone B (detoxification), the textual results preferably indicate a state ranging from normal function to markedly overloaded.

In Zone C (hormonal-adrenal), the textual results preferably indicate a state ranging from significant hyperfunction to normal to significant hypofuntion.

With respect to the central nervous system (CNS), textual conclusions, based on the measured base omega potentials (discussed above) are also preferably generated. These include conclusions that address the state of adaptive reaction of the CNS (ranging from adequate to a restriction in the effectiveness and quality of the adaptation reaction), resistance of CNS to physical and psychic loads (ranging from satisfactory to low resistance) and level of activity of CNS (ranging from optimal to low).

### Electromyocardiogram (EMG)

Referring to FIG. 9, a flowchart for a representative EMG assessment in accordance with the present invention is shown. FIG. 10A-10D illustrates electrode placement on various muscles, while FIG. 11 illustrates a sensed EMG signals and calculation of LTC and LTR from the EMG signal.

The EMG assessment may utilize the parameters of Latent Time to Muscle Contraction (LTC) which is the time (measured in seconds) between a muscle contraction signal from MD 60 and the detection of contractions in the sensed EMG data and Latent Time to Muscle Relaxation (LTR) which is the time between a muscle relaxation signal from MD 60 and the detection of muscle relaxation (absence of contractions) in the sensed EMG data. The moment of the muscle contraction is determined by the presence of the EMG signal at the channel amplifier (in the transmission pod 50) while the moment of relaxation is determined by the absence of the EMG signal at the channel amplifier.

For an EMG test, a SUT may be prompted by a MD 60 for the type of sport or physical activity that the SUT is training for, step 210. Different physical activities use different muscles. For example, for runners, cyclists and speed skaters, the right or left thigh muscle is recommended. Attachment to the left thigh muscle is shown in FIG. 2. FIGS. 10A-10D illustrate electrode placement for bicep, tricep, quadricep and hamstring based assessment, respectively. MD 60 responds with instructions for muscle and electrode placement (step 212). Is the electrode placed correctly and the SUT ready (step 214)?

The SUT is instructed to quickly and heavily strain the researched muscle after a first signal, sound/light/other, is given and then to relax that muscle as soon as a second signal is given. EMG data is received and assessed (as shown in FIG. 11) to generate LTC and LTR measurements, step 216 and 218, respectively.

This sequence of stimuli, contraction and relaxation is repeated a set number of times, typically 7-10 times, with the time of contraction varying randomly between 5-8 seconds and the rest between 15-20 seconds. Data from each contraction interval is held (step 218) and a determination is made as to whether the desired number of trials has been completed (step 220).

When the requisite series of trials is done, the average LTC and LTR are preferable calculated (step 224) and an index of neuromuscular adaptation (INA) is generated as LTC/LTR (step 226). There is considerable variation in these three parameters based on gender, age, sports specialization and level of physical activity. They may range normally as LTC: 180-325 millisecond (ms), LTR: 150-220 ms, INA or K: 0.8-2.2, or beyond in more extreme instances. The smaller the INA value the more fatigued the subject muscle. Representative values for a given assessment might be LTC: 273, LTR: 180 and INA: 1.52, which would indicate a condition on the cusp of Slight Tiredness and Incomplete Recovery (see below). These parameters have been studied by Fedorov, V. L. and are discussed in Fedorov, V. L., “EMG Registration Latency Time for Voluntary Contraction and Relaxation of Skeletal Muscles as a Method of Evaluating the Functional State of the Neuromuscular System of the Athlete,” Commission on the Physiology of Sport, Kiev, 1957, pp. 143-144.

Processing logic 70 preferably converts these parameters to a pre-defined textual or graphic conclusion of the functional state of the assessed muscle. The textual conclusion derived from the parameters may be one of the following.

Current State of Muscle System is Characterized as:

State of Complete Recovery—
Readiness for workout is high.

State of Incomplete Recovery—
Readiness for workout is average. Developmental physical exercise not recommended.

State of Slight Tiredness—
Readiness for workout is below average. Rest recommended.

State of Exhaustion—
Readiness for workout is low. Recovery procedure is preferred.
   “Developmental” exercise refers to those activities that induce muscle fatigue and break down the muscle to trigger subsequent growth during rest. In State of Incomplete Recovery, stimulus exercise, light exercise that does induce significant fatigue, is recommended. An example of stimulus exercise would be a slower run or jog in place of a hard run. “Recovery procedure” refers to acupuncture, massage or other therapies known to restore muscle tissues. Preferably, the recovery procedures go beyond mere rest.

### Overall Assessment

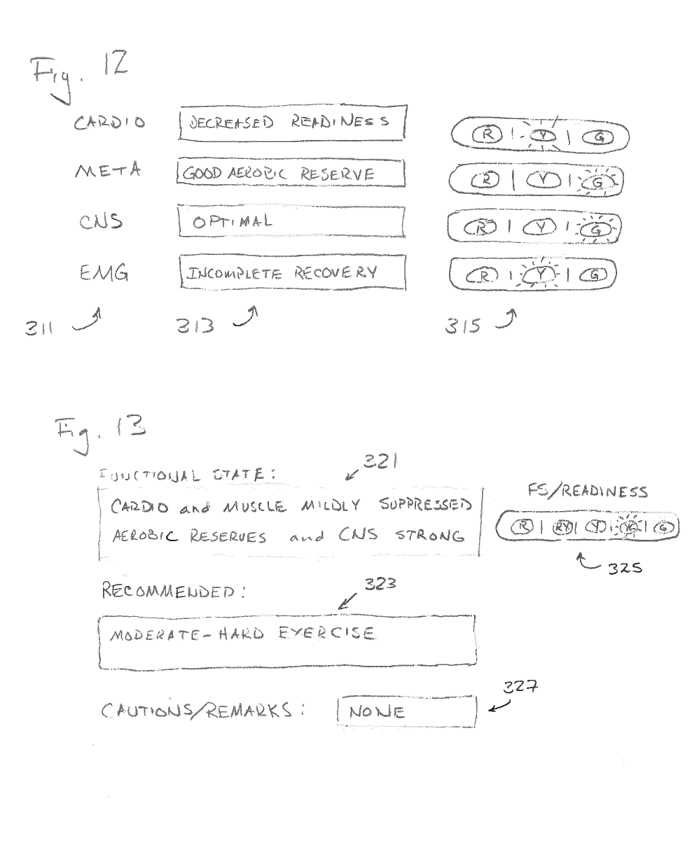

Processing logic 70 may generate various reports and analyses which may be accessed by mobile device 60 and/or 3P computer 80. These reports may be detailed or simplified depending on the requests of the user. FIG. 12 illustrates one embodiment of a simplified presentation of results for various body system assessments. Fields may include name of body system 311, brief conclusion and/or recommendation 313 and a visual indicator of readiness/state 315. FIG. 12 illustrates Cardio, Metabolic, Central Nervous System (CNS) and Muscular body systems entries 311. A textual conclusion of “state”, which may be accompanied by a recommendation for level of exercise, is preferably presented adjacent the body system headers. To the right, a visual indicator 315, similar to a horizontal traffic light, is presented for each system. The Red signifying rest or “stop” exercise, the Yellow meaning exercise but with certain limitations and the Green meaning “go” exercise.

The break out of these body systems test results gives a user greater awareness of how his or her individual body systems are fairing, which inherently gives the SUT greater physical self-awareness.

The assessment results may also be presented as a combined result. One such embodiment is shown in FIG. 13. This display, suitable for viewing on MD 60 or 3P computer 80, may include a functional state report field 321, a recommended activity field 323, a visual indicator field 325 (for example the horizontal traffic light of FIG. 12 though perhaps with more lights in the spectrum, e.g., red-yellow and yellow-green or more/others added), and a caution, warning or remarks field 327 where important information may be displayed.

A representative functional state conclusion is provided at 321, as is the recommended activity level 323. The light for yellow-green (YG) is light in 325 and “None” is in the Caution/Remarks field 327. Should a SUT's body system assessments undercover a condition of concern, it would be noted in the Caution/Remarks field.

While the invention has been described in connection with specific embodiments thereof, it will be understood that it is capable of further modification, and this application is intended to cover any variations, uses, or adaptations of the invention following, in general, the principles of the invention and including such departures from the present disclosure as come within known or customary practice in the art to which the invention pertains and as may be applied to the essential features herein before set forth, and as fall within the scope of the invention and the limits of the appended claims.

1. An apparatus for assessing the physical functional state and preparedness for exertion of body systems of a subject under test (SUT), comprising:
interface logic adapted to execute on a mobile device and to receive through that mobile device sensed EMG data from a SUT and at least one of sensed brain wave and sensed cardiac data from that SUT, the interface logic transmitting the sensed EMG data and one of said brain wave or cardiac data from that mobile device; and
assessment logic, located at a distance from a mobile device executing the interface logic, that:
(1) receives the sensed EMG data transmitted by the interface logic and conducts an assessment procedure to determine the functional state of skeletal muscles of the SUT based on the received EMG data; and
(2) receives the sensed brain wave or sensed cardiac data transmitted by the interface logic and conducts a brain wave assessment procedure or a cardiac assessment procedure, respectively, to determine the functional state of a body system that corresponds to the one of the brain wave or cardiac data received by the assessment logic;
wherein the assessment logic generates from the EMG procedure an EMG test result signal that represents the functional state of a skeletal muscle of that SUT; and
wherein the assessment logic generates from the one of the brain wave and cardiac procedures that was conducted a brain wave or a cardiac test result signal, respectively, that represents the functional state of a corresponding body system of that SUT.

2. The apparatus of claim 1, wherein the interface logic is adapted to receive both sensed brain wave data and sensed cardiac data from the SUT;
wherein the assessment logic receives both sensed brain wave data and sensed cardiac data from the interface logic and conducts both the brain wave assessment procedure and the cardiac data assessment procedure; and
wherein the assessment logic further generates both a brain wave test result signal that represents the functional state of a body system that corresponds to the brain wave procedure and a cardiac test result signal that represents the functional state of a body system that corresponds to the cardiac procedure.

3. The apparatus of claim 1, further comprising transmission logic that transmits the EMG test signal and the one of the brain wave and cardiac test signals to a mobile device on which the interface logic is executing.

4. The apparatus of claim 1, further comprising transmission logic that transmits the EMG test signal and the one of the brain wave and cardiac test signals to a third party computer located at a distance from the assessment logic.

5. The apparatus of claim 1, wherein the assessment logic is cloud-based and is capable of connecting through one or more of a mobile device network and the internet to the mobile device or a third party computer located at a distance from the assessment logic.

6. The apparatus of claim 1, wherein the cardiac assessment procedure includes one or more of HRV, ECG and DECG based assessment, and further wherein the interface logic is adapted to receive 2-lead DECG based data from a SUT, and the assessment logic is configured to conduct a DECG based cardiac assessment procedure using the 2-lead DECG data received by the interface logic and transmitted to the assessment logic.

7. The apparatus of claim 1, wherein the brain wave assessment procedure includes receiving and assessing omega brain wave data from a SUT.

8. The apparatus of claim 1, wherein the assessment logic is configured for communication with a third party computer located at a distance to permit the third party computer to (a) access at least one of received sensed data and test result signals and (b) modify one or more of the EMG, brain wave and cardiac assessment procedures.

9. The apparatus of claim 1, wherein the interface logic is configured to execute on a mobile device in a manner that permits a SUT to select an EMG based assessment and one or more of a brain wave based assessment and a cardiac based assessment, and further that the interface logic collects data for the assessments selected by the SUT and forwards this data to the assessment logic for determination of function state of corresponding body systems.

10. The apparatus of claim 1, further comprising a SUT borne transmitter device that includes multiple channels for respectively propagating bio-signal data from EMG, brain wave and cardiac specific sensors.

11. The apparatus of claim 1, wherein the EMG and the brain wave or cardiac test result signals represent at least one of a textual and a graphic conclusion about the functional state of their corresponding body systems.

12. An apparatus for assessing the physical functional state and preparedness for exertion of body systems of a subject under test (SUT), comprising:
interface logic adapted to execute on a mobile device and to receive through that mobile device sensed EMG data from a SUT and sensed cardiac data from that SUT, the interface logic transmitting the sensed EMG data and the sensed cardiac data from that mobile device; and
assessment logic, located at a distance from a mobile device executing the interface logic, that:
(1) receives the sensed EMG data transmitted by the interface logic and conducts an assessment procedure to determine the functional state of skeletal muscles of the SUT based on the received EMG data; and
(2) receives the sensed cardiac data transmitted by the interface logic and conducts a cardiac assessment procedure to determine the functional state of a corresponding body system of the SUT based on the received cardiac data;
wherein the assessment logic generates from the EMG procedure an EMG test result signal that represents the functional state of a skeletal muscle of that SUT; and
wherein the assessment logic generates from the cardiac procedure a cardiac test result signal that represents the functional state of a corresponding body system of that SUT.

13. The apparatus of claim 12, further comprising transmission logic coupled to the assessment logic that transmits the EMG test result signal and the cardiac test result signal to at least one of a mobile device on which the interface logic is executing and a third party computer located at a distance from the assessment logic.

14. The apparatus of claim 12, wherein the cardiac assessment procedure includes 2-lead DECG based assessment, and further wherein the interface logic is adapted to receive 2-lead DECG based data from a SUT, and the assessment logic is configured to conduct a DECG based cardiac assessment procedure using the 2-lead DECG data received by the interface logic and transmitted to the assessment logic.

15. The apparatus of claim 12, further comprising a multi-channel user-borne transmitter for transmission of sensed bio-signals from a SUT to the interface logic.

16. The apparatus of claim 12, wherein the assessment logic generates an electronic signal representative of a textual or graphical conclusion of a combined assessment result for the functional state of a SUT, based on the assessment of multiple body system of that SUT.

17. The apparatus of claim 12, wherein the assessment logic generates an electronic signal representative of a textual or graphical display of a SUT's overall readiness for physical activity, based on assessment of multiple body systems, and an electronic signal of recommended exercise level based on that assessment of multiple body systems.

18. An apparatus for assessing the physical functional state and preparedness for exertion of body systems of a subject under test (SUT), comprising:
interface logic adapted to execute on a mobile device and to receive through that mobile device sensed EMG data, sensed brain wave data and sensed cardiac data from a SUT, the interface logic transmitting the sensed EMG, brain wave and cardiac data from that mobile device; and
assessment logic, located at a distance from a mobile device executing the interface logic, that:
(1) receives the sensed EMG data transmitted by the interface logic and conducts an assessment procedure to determine the functional state of skeletal muscles of the SUT based on the received EMG data;
(2) receives the sensed brain wave data transmitted by the interface logic and conducts a brain wave assessment procedure to determine the functional state of a body system that corresponds to the brain wave data received by the assessment logic; and
(3) receives the sensed cardiac data transmitted by the interface logic and conducts a cardiac assessment procedure to determine the functional state of a body system that corresponds to the cardiac data received by the assessment logic;
wherein the assessment logic generates from the EMG procedure an EMG test result signal that represents the functional state of a skeletal muscle of that SUT; and
wherein the assessment logic generates from the brain wave procedure a brain wave test result signal that represents the functional state of a corresponding brain wave body system and from the cardiac procedure a cardiac test result signal that represents the functional state of a corresponding cardiac body system.

19. The apparatus of claim 18, wherein the assessment logic processes one or more of omega brain wave sensed from the SUT and 2-lead DECG-based data sensed from the SUT.

20. The apparatus of claim 18, wherein the assessment logic generates at least one of an electronic signal representative of a textual or graphical display of a SUT's overall readiness for physical activity, based on assessment of multiple body systems, and an electronic signal of recommended exercise level based on that assessment of multiple body systems.
